(19) United States
(12) Patent Application Publication
(10) Pub. No.: US 2025/0114183 A1
(43) Pub. Date: Apr. 10, 2025

(54) ARTIFICIAL BLOOD VESSEL PROVIDED WITH PIEZOELECTRIC VIBRATION STRUCTURE

(71) Applicant: IUCF-HYU (INDUSTRY-UNIVERSITY COOPERATION FOUNDATION HANYANG UNIVERSITY), Seoul (KR)

(72) Inventors: Jin Pyo HONG, Seoul (KR); Woo Jong KIM, Seoul (KR)

(21) Appl. No.: 18/579,510

(22) PCT Filed: Jul. 1, 2022
(86) PCT No.: PCT/KR2022/009534

§ 371 Date: Aug. 13, 2024

(30) Foreign Application Priority Data
Jul. 19, 2021 (KR) 10-2021-0094104

(51) Int. Cl. A61F 2/06; A61B 17/00; A61B 17/22; A61L 27/44; A61L 27/50

(52) U.S. Cl. CPC A61F 2/06; A61B 17/22012; A61L 27/446; A61L 27/507; A61B 2017/00402; A61F 2250/0093

(57) ABSTRACT

One embodiment of the present invention relates to an artificial blood vessel installed with a piezoelectric vibration element. In the artificial blood vessel according to one embodiment of the present invention, a piezoelectric vibration element is installed at one or both ends of a polymer tube, and vibration occurs in the piezoelectric vibration element according to an electrical signal. As a result, the formation of blood clots or the overgrowth of the intima can be prevented at the anastomotic site where the artificial blood vessel and the native blood vessel are joined, thereby improving the vascular patency rate and preventing stenosis.

### TECHNICAL FIELD

Example embodiments of the present invention relate to artificial blood vessels, and more specifically to artificial blood vessels equipped with piezoelectric vibration elements.

### BACKGROUND ART

Due to the population aging, the number of patients with various vascular diseases accompanied by blood flow disorders caused by narrowed or blocked blood vessels due to cholesterol deposition on the endothelium of human blood vessels and proliferation of endothelial cells. Diseases related to atherosclerosis, commonly known as arteriosclerosis, include myocardial infarction, cerebral infarction, and obstructive peripheral artery disease (PAD). In particular, obstructive peripheral vascular disease is known to be prone to recurrence and difficult to cure. Conventional techniques for dilating narrowed blood vessels include percutaneous transluminal angioplasty (PTA), stenting, and arterial bypass surgery. Arterial bypass surgery requires autologous or artificial blood vessels, and autologous blood vessels are only available through the great saphenous veins of both legs, so the scope of the surgery is limited. In particular, when using small-diameter artificial blood vessels with a diameter of 6 mm or less, problems such as thrombosis and neointimal hyperplasia occur at a high rate.

### DISCLOSURE

### Technical Problem

The technical object of the present invention is to provide an artificial blood vessel for improving blood vessel patency rate by suppressing thrombus formation and intimal proliferation at the anastomosis site with blood vessels.

The technical problems of the present invention are not limited to the technical problems mentioned above, and other technical problems not mentioned will be clearly understood by those skilled in the art from the description below.

### Technical Solution

In order to solve the above problems, the present invention may provide an artificial blood vessel. The artificial blood vessel includes a polymer tube and a piezoelectric vibration element disposed at one or both ends of an outer wall of the polymer tube. The piezoelectric vibration element includes a first electrode disposed on the outer wall of the polymer tube, a polymer piezoelectric layer disposed on the first electrode, and a second electrode disposed on the polymer piezoelectric layer.

The piezoelectric vibration element may generate vibration by receiving electricity.

The polymer tube may be a thermoplastic polymer, such as Darcon (woven polyethylene terephthalate), ePTFE (expanded poly-tetrafluoroethylene), PU (polyurethane), PLA (polylactic acid), PGA (polyglycolic acid), PLGA (poly(lactic-co-glycolic acid)), SIS(poly(styrene-isoprene-styrene)), SBS(poly(styrene-butadiene-styrene)), SIBS(poly(styrene-isobutylene-styrene)), SEBS(poly(styrene-ethylene-butylene-styrene)) or a mixture of one or more of these.

The polymer piezoelectric layer may be made of a polymer piezoelectric material or a polymer composite piezoelectric material.

The polymer piezoelectric material may be at least one selected from a group consisting of polyvinylidene fluoride (PVDF), polyvinylidene fluoride-trifluoroethylene (P(VDF-TrFE)), polyvinylidene fluoride-tetrafluoroethylene (P(VDF-TeFE)), polyvinylidene fluoride-trifluoroethylene-chlorotrifluoroethylene (P(VDF-TrFE-CTFE)), polyvinylidene fluoride-trifluoroethylene-chlorofluoroethylene (P(VDF-TrFE-CFE)), triglycine sulfate (TGS), and mixtures thereof.

The polymer composite piezoelectric material may be a mixture of a polycrystalline piezoelectric material and the polymer piezoelectric material.

The polycrystalline piezoelectric material may be at least one selected from BaTiO₃, ZnO, KNbO₃, NaNbO₃, CaTiO₃, Bi_{0.5}Na_{0.5}TiO₃, KSr₂Nb₅O₁₅, and mixtures thereof.

### Advantageous Effects

According to the present invention described above, the artificial blood vessel according to the present invention may suppress thrombus formation and intimal proliferation at the anastomosis site with the blood vessel and improves blood vessel patency by generating vibration in the piezoelectric vibration element installed at one or both ends of the artificial blood vessel.

### DESCRIPTION OF DRAWINGS

FIG. 1 is a perspective view of an artificial blood vessel equipped with a piezoelectric vibration element according to an embodiment of the present invention.

FIG. 2(a) is a schematic diagram showing an anastomosis site after an artificial blood vessel and a blood vessel are joined, and FIG. 2(b) is a schematic diagram showing the anastomosis site after an artificial blood vessel with a piezoelectric vibration element and a blood vessel are joined according to the present invention.

### MODES OF THE INVENTION

The present invention can be subject to various changes and can have various forms, and specific embodiments will be illustrated in the drawings and described in detail in the text. However, this is not intended to limit the present invention to a specific disclosed form, and should be understood to include all changes, equivalents, and substitutes included in the spirit and technical scope of the present invention. While describing each drawing, similar reference numerals are used for similar components.

Unless otherwise defined, all terms used herein, including technical or scientific terms, have the same meaning as commonly understood by a person of ordinary skill in the technical field to which the present invention pertains. Terms defined in commonly used dictionaries should be interpreted as having a meaning consistent with the meaning in the context of the related technology, and should not be interpreted in an ideal or excessively formal sense unless explicitly defined in the present application.

Hereinafter, preferred embodiments of the present invention will be described in more detail with reference to the attached drawings.

### Artificial Blood Vessel

An artificial blood vessel equipped with a piezoelectric vibration element according to an embodiment of the present invention includes a polymer tube and a piezoelectric vibration element attached to the polymer tube and containing a polymer piezoelectric material that exhibits an inverse piezoelectric effect when receiving an external electrical signal, so that, when electricity is applied to the piezoelectric vibration element, vibration is generated in the piezoelectric vibration element due to the applied electricity, and the vibration can be transmitted to the polymer tube adjacent to the piezoelectric vibration element.

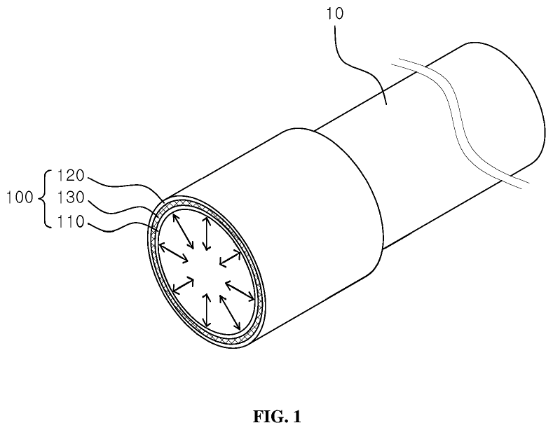

FIG. 1 is a perspective view of an artificial blood vessel equipped with a piezoelectric vibration element according to an embodiment of the present invention.

Referring to FIG. 1, the artificial blood vessel on which the piezoelectric vibration element is installed according to an embodiment of the present invention may include a polymer tube 10.

The polymer tube 10 may include a thermoplastic polymer material, and the thermoplastic polymer may be a polymer material that has excellent biocompatibility due to no immune rejection, has high stretchability and high elasticity properties, and is capable of extrusion molding. For example, the thermoplastic polymer material may be polyethylene terephthalate (PET), expanded poly-tetrafluoroethylene (ePTFE), polyurethane (PU), polylactic acid (PLA), polyglycolic acid (PGA), poly(lactic-co-glycolic acid) (PLGA), poly(styrene-isoprene-styrene)) (SIS), poly(styrene-butadiene-styrene) (SBS), poly(styrene-isobutylene-styrene) (SIBS), poly(styrene-ethylene-butylene-styrene) (SEBS), or a mixture of one or more of these, but is not limited to this.

The polymer tube 10 may be a small-diameter tube with, for example, a diameter of 5 mm or less or a submillimeter diameter, or a mid-to a large-diameter tube with millimeter diameter, for example, a diameter ranging from 6 to 10 mm, but it is not limited thereto. In particular, when a small-diameter polymer tube is used as an artificial blood vessel, blood flow rate therein may be low and blood clots can easily form, so that relatively better effects can occur when the piezoelectric vibration element is installed on the small-diameter tube with the diameter of 5 mm or less.

The length of the polymer tube 10 may be several centimeters to several meters, for example, 1 cm to 50 m, but is not limited thereto.

The polymer tube 10 may be manufactured by an extrusion molding.

A blood coagulation inhibitor such as an anticoagulant or an antiplatelet agent or a smooth muscle cell proliferation inhibitor may be additionally attached to the inner and/or outer walls of the polymer tube 10. For example, among the blood coagulation inhibitors, the anticoagulant may be heparin, hirudin, argatroban, thrombomodulin, etc., and the antiplatelet agent may be aspirin., abciximab, nitric oxide (NO), etc., and the smooth muscle cell proliferation inhibitor may be paclitaxel, but is not limited thereto.

In order to attach drugs such as blood coagulation inhibitors to the inner and/or outer walls of the polymer tube 10, the inner and/or outer walls of the polymer tube 10 may be additionally subjected to surface treatment, such as drug immersion treatment, plasma treatment, etc., but is not limited to this.

In addition, the artificial blood vessel installed with the piezoelectric vibration element according to an embodiment of the present invention may include the piezoelectric vibration element 100 installed at one or both ends of the outer wall of the polymer tube 10. In detail, considering that anastomosis with native blood vessels occurs at at least one, and usually at least two, position(s) of the polymer tube 10, one or two piezoelectric vibration elements 100 may be installed.

The piezoelectric vibration element 100 may be attached to the outer wall of the polymer tube 10 and may be disposed in a shape that surrounds at least a portion of the outer wall of the polymer tube 10, for example, a cylindrical shape, a semi-cylindrical shape, etc. The length of the piezoelectric vibration element 100 may be shorter than the length of the polymer tube 10, so that the outer wall surface of the polymer tube 10 may be exposed to the outside.

The piezoelectric vibration element 100 may include a first electrode 110 disposed on the outer wall of the polymer tube 10, a polymer piezoelectric layer 130 disposed on the first electrode 110, and a second electrode 120 disposed on the polymer piezoelectric layer 130. In other words, the piezoelectric vibration element 100 may have the first electrode 110 and the second electrode 120 combined to both sides of the polymer piezoelectric layer 130.

First, the first electrode 110 may be disposed between the outer wall of the polymer tube 10 and the polymer piezoelectric layer. Additionally, the second electrode 120 may be disposed on the opposite side of the polymer piezoelectric layer 130 to which the first electrode 110 is disposed. The first electrode 110 and the second electrode 120 may be connected to wires (not shown), for example, metal wires that transmit electricity from an external electricity generating device (not shown). As a result, electricity, for example, an alternating current voltage, may be applied to the, the polymer piezoelectric layer 130.

The first electrode 110 and/or the second electrode 120 may be a metal thin film having electrical conductivity for applying an alternating current voltage to the polymer piezoelectric layer 120. The material used as the metal thin film may have little or no biotoxicity and may be, for example, silver, gold, platinum, copper, aluminum, nickel, titanium, etc., but are not limited thereto.

The thickness of the first electrode 110 and/or the second electrode 120 may be a nano-to micro-sized thin film, for example, 1 nm to 1000 μm, but is not limited thereto and may be adjusted appropriately depending on experimental conditions, etc.

To form the first electrode 110 and/or the second electrode 120, various methods, for example, sol-gel synthesis, paste application, sputtering, and evaporation, physical vapor deposition (PVD), chemical vapor deposition (CVD), atomic layer deposition (ALD), or similar methods may be used, but are not limited thereto.

The surface of the polymer tube 10 may be additionally subjected to surface treatment, for example, plasma treatment. Alternatively, an adhesive, such as epoxy or PMMA, may be additionally applied between the polymer tube 10 and the first electrode 110, but is not limited thereto. In this case, the first electrode 110 may be closely adhered to the outer wall of the polymer tube 10. Additionally, the surface of the polymer piezoelectric layer 130 may be additionally subjected to surface treatment, for example, plasma treatment, but is not limited thereto. In this case, the second electrode 120 may be closely adhered to the surface of the polymer piezoelectric layer 130.

In the piezoelectric vibration element 100, when an alternating current voltage is applied from an external electricity generating device (not shown) to the first electrode 110 and the second electrode 120, the polymer piezoelectric layer 120 may cause vibration.

The polymer piezoelectric layer 120 may be a material with ferroelectric properties, and may exhibit an inverse piezoelectric effect in which strain is generated when voltage is applied to the material, and may convert electrical energy into mechanical energy to cause mechanical displacement. When the mechanical displacement appears as vibration, the amplitude of the vibration can be controlled by adjusting the magnitude of the applied alternating current voltage, and conversely, the external vibration signal can be converted into an electric signal.

The vibration may be ultrasonic vibration. Specifically, the polymer piezoelectric layer 120 may be capable of low-voltage driving in the mid-frequency range of ultrasonic waves, for example, the frequency range of 200 to 600 Hz, but is not limited thereto, and the range of the alternating current voltage and frequency can be determined through experiment conditions, etc.

The vibration may mean that the mechanical displacement of the piezoelectric layer, for example, contraction and expansion, occurs repeatedly one or more times, and the direction of the vibration is radial with respect to the direction in which the polymer tube extends, as shown by the arrow in FIG. 1. Accordingly, a micro-displacement may be generated due to the vibration generated in the piezoelectric vibration element 100, and the generated vibration may be transmitted to the polymer tube 10 adjacent to the piezoelectric vibration element 100.

The generated vibration can prevent the formation of a blood clot in the blood vessel adjacent to the anastomosis site, dislodge the generated blood clot, or prevent overgrowth of the intima.

The polymer piezoelectric layer 120 may be formed of a film-type polymer piezoelectric material in a single layer or as a multilayer of two or more layers.

The polymer piezoelectric layer 120 has a thin thickness and elastic flexibility, and has an acoustic impedance similar to that of biological tissue, so it has good impedance matching for medical use. The polymer piezoelectric layer 120 may include a polymer piezoelectric material or a composite piezoelectric material that is a composite of the polymer piezoelectric material and a polycrystalline piezoelectric material.

The polymer piezoelectric material may be, for example, at least one selected from polyvinylidene fluoride (PVDF), polyvinylidene fluoride-trifluoroethylene (P(VDF-TrFE)), polyvinylidene fluoride-tetrafluoroethylene (P(VDF)-TeFE)), polyvinylidene fluoride-trifluoroethylene-chlorotrifluoroethylene (P(VDF-TrFE-CTFE)), polyvinylidene fluoride-trifluoroethylene-chlorofluoroethylene (P(VDF-TrFE-CFE)), triglycine sulfate (TGS), and mixtures thereof, but is not limited thereto.

The composite piezoelectric material may be a composite of the polycrystalline piezoelectric material and the polymer piezoelectric material. For example, the polycrystalline piezoelectric material may be at least one selected from BaTiO₃, ZnO, KNbO₃, NaNbO₃, CaTiO₃, Bi_{0.5}Na_{0.5}TiO₃, KSr₂Nb₅O₁₅, and mixtures thereof, but is not limited thereto.

The polymer piezoelectric layer 120 may be additionally subjected to a poling process and/or a biaxial elongation process to improve piezoelectric performance.

The polymer piezoelectric layer 120 may be formed using, for example, dip coating, spray coating, aerosol coating, spin coating, inkjet printing, solution casting, or similar methods. As another example, the polymer piezoelectric layer 120 may be made of a commercially available polymer piezoelectric film, for example, Kynar©.

The polymer piezoelectric layer 120 may be a single layer or a multi-layer of two or more layers of a micro-sized thin film. The thickness of the single layer may be, for example, 1 to 1000 μm, but is not limited thereto.

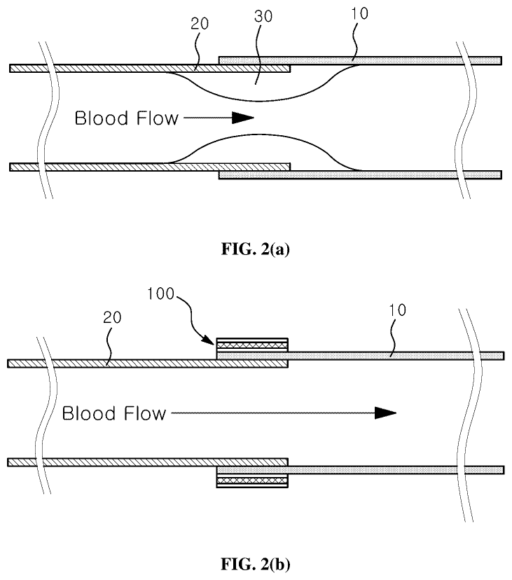

FIG. 2(a) is a schematic diagram showing an anastomosis site after the artificial blood vessel and a native blood vessel are joined, and FIG. 2(b) is a schematic diagram showing the anastomosis site after the artificial blood vessel with the piezoelectric vibration element according to the present invention and the native blood vessel are joined.

Referring to FIG. 2(a), one end of the polymer tube 10 used as an artificial blood vessel may be inserted into or outside of one end of the native blood vessel 20, or may be connected without overlapping. For example, if the inner diameter of the polymer tube 10 is larger than the outer diameter of the blood vessel 20, a portion of the blood vessel 20 may be inserted into the polymer tube 10 and anastomosed with the polymer tube 10. Meanwhile, if the outer diameter of the polymer tube 10 is smaller than the inner diameter of the blood vessel 20, a part of the polymer tube 10 may be inserted into the blood vessel 20 and anastomosed with the the blood vessel 20. On the other hand, when the inner diameter of the polymer tube 10 is the same or similar to the inner diameter of the blood vessel 20, one end of the polymer tube 10 and one end of the blood vessel 20 are anastomosed in parallel without overlapping.

After the polymer tube 10 is anastomosed with the blood vessel 20 and implanted into a living body, a thrombus or intima 30 may be formed at the anastomosis site. In other words, platelets in the blood may aggregate at the anastomosis site to form a thrombus 30, which may limit blood flow. In addition, at the anastomosis site, smooth muscle cells may excessively proliferate in the intima and the amount of extracellular matrix (ECM) may increase, which may thicken the intima 30, which may result in limited blood flow or stenosis of blood vessels.

On the other hand, referring to FIG. 2(b), the artificial blood vessel of the present invention includes a piezoelectric vibration element 100 installed on the outer wall of one end of the polymer tube 10, and can prevent the formation of a blood clot or intima at the anastomosis site.

One end of the polymer tube 10 on which the piezoelectric vibration element 100 is installed may be overlapped on the outside or inside of one end of the blood vessel 20 and may be anastomosed with the blood vessel 20, or may be anastomosed without overlapping. For example, when the inner diameter of the polymer tube 10 on which the piezoelectric vibration element 100 is installed is larger than the outer diameter of the blood vessel 20, a portion of the blood vessel 20 may be inserted into the polymer tube 10 on which the piezoelectric vibration element 100 is installed and anastomosed with the polymer tube 10. Meanwhile, if the outer diameter of the polymer tube 10 is smaller than the inner diameter of the blood vessel 20, a part of the polymer tube 10 on which the piezoelectric vibration element 100 is installed may be inserted into the blood vessel 20 and anastomosed with the the blood vessel 20. On the other hand, when the inner diameter of the polymer tube 10 on which the piezoelectric vibration element 100 is installed is the same or similar to the inner diameter of the blood vessel 20, one end of the polymer tube 10 and one end of the blood vessel 20 are anastomosed in parallel without overlapping. However, if the inner diameter of the polymer tube 10 on which the piezoelectric vibration element 100 is installed is larger than the outer diameter of the blood vessel 20, and a part of the blood vessel 20 is inserted into the polymer tube 10 and anastomosed with the polymer tube 10, at the anastomosis site, blood flow may not encounter a step or ridge due to a decrease in the diameter of the blood vessel. As a result, since the generation of vortices at the anastomosis site is suppressed, the formation of blood clots or intima can be better suppressed.

During anastomosis with the blood vessel 20, a needle hole passing through the piezoelectric vibration element 100 and the polymer tube 10 may be created in the artificial blood vessel on which the piezoelectric vibration element 100 of the present invention is installed by a surgical needle, etc. However, this may not be a problem in the operation of the piezoelectric vibration element 100.

The piezoelectric vibration element 100 is electrically connected to an external power supply (not shown) and can receive an alternating current voltage therefrom to generate vibration, for example, ultrasonic vibration. Accordingly, physical stimulation can be repeatedly applied to the anastomosis site with the blood vessel 20. The repetitive and stimulating mechanical movement generated by the piezoelectric vibration element 100 can fundamentally inhibit platelet aggregation and smooth muscle cell proliferation at the anastomosis site. Accordingly, the formation of blood clots can be prevented and overgrowth of the intima can be suppressed.

Meanwhile, the embodiments of the present invention disclosed in the specification and drawings are merely provided as specific examples to aid understanding, and are not intended to limit the scope of the present invention. It is obvious to those skilled in the art that in addition to the embodiments disclosed herein, other modifications based on the technical idea of the present invention can be implemented.

1. An artificial blood vessel comprising:
a polymer tube; and
a piezoelectric vibration element disposed at one or both ends of the outer wall of the polymer tube and including a first electrode disposed on the outer wall of the polymer tube, a polymer piezoelectric layer disposed on the first electrode, and a second electrode disposed on the polymer piezoelectric layer.

2. The artificial blood vessel of claim 1, wherein the piezoelectric vibration element generates vibration by receiving electricity.

3. The artificial blood vessel of claim 1, wherein the polymer tube is a thermoplastic polymer selected from a group consisting of PET(polyethylene terephthalate), ePTFE (expanded poly-tetrafluoroethylene), PU (polyurethane), PLA (polylactic acid), PGA (polyglycolic acid), PLGA (poly(lactic-co-glycolic acid)), SIS(poly(styrene-isoprene-styrene)), SBS(poly(styrene-butadiene-styrene)), SIBS(poly(styrene-isobutylene-styrene)), SEBS(poly(styrene-ethylene-butylene-styrene)) and a mixture of one or more of these.

4. The artificial blood vessel of claim 1, wherein the polymer piezoelectric layer is made of a polymer piezoelectric material or a polymer composite piezoelectric material.

5. The artificial blood vessel of claim 4, wherein the polymer piezoelectric material is at least one selected from a group consisting of polyvinylidene fluoride (PVDF), polyvinylidene fluoride-trifluoroethylene (P(VDF-TrFE)), polyvinylidene fluoride-tetrafluoroethylene (P(VDF-TeFE)), polyvinylidene fluoride-trifluoroethylene-chlorotrifluoroethylene (P(VDF-TrFE-CTFE)), polyvinylidene fluoride-trifluoroethylene-chlorofluoroethylene (P(VDF-TrFE-CFE)), triglycine sulfate (TGS), and mixtures thereof.

6. The artificial blood vessel of claim 4, wherein the polymer composite piezoelectric material is a mixture of a polycrystalline piezoelectric material and the polymer piezoelectric material.

7. The artificial blood vessel of claim 6, wherein the polycrystalline piezoelectric material is at least one selected from BaTiO₃, ZnO, KNbO₃, NaNbO₃, CaTiO₃, Bi_{0.5}Na_{0.5}TiO₃, KSr₂Nb₅O₁₅, and mixtures thereof.
